(19) United States
(12) Patent Application Publication
(10) Pub. No.: US 2016/0328813 A1
(43) Pub. Date: Nov. 10, 2016

(54) MEDICAL SUPPLIES CONTAINER AND RELATED SYSTEMS

(71) Applicant: INVENTORY TECHNOLOGIES LIMITED, Christchurch (NZ)

(72) Inventors: Peter James MONTGOMERY, Kaiapoi (NZ); Peter GILLMAN, Kaiapoi (NZ)

(21) Appl. No.: 15/093,654

(22) Filed: Apr. 7, 2016

Related U.S. Application Data
(63) Continuation-in-part of application No. PCT/IB2014/065097, filed on Oct. 7, 2014.

(30) Foreign Application Priority Data
Oct. 7, 2013 (NZ) 616354
Nov. 21, 2013 (NZ) 618095

(51) Int. Cl. G06Q 50/22; G06Q 10/08

(52) U.S. Cl. CPC G06Q 50/22; G06Q 10/087

(57) ABSTRACT

A monitored dispensary for medical items comprising or including a storage device to which multiple users have access, said storage device having a containment region inside of which items are stored, and a monitoring system, said monitoring system comprising detectors to obtain information about the items used during a transaction when a user accesses the cabinet, and devices to obtain identity information derived from the user of the cabinet at the time that the transaction was effected, wherein said system is able to process the output of the detectors and/or devices in order to create a record of each transaction and monitor the inventory of the storage device.

### CROSS-REFERENCE TO RELATED APPLICATIONS

This application is a Continuation-in-Part of International Patent Application No. PCT/IB2014/065097, filed Oct. 7, 2014, which claims the benefit of New Zealand Patent Application No. 616354, filed Oct. 7, 2013, and New Zealand Patent Application No. 618095, filed Nov. 21, 2013, each of which is hereby incorporated by reference in its entirety.

### FIELD OF THE INVENTION

The present invention relates to a medical supplies container in a particular, although not solely to a medical supplies container, that may be made available for limited or open use by individuals requiring, for example, first aid supplies at industrial, commercial or medical facilities.

### BACKGROUND TO THE INVENTION

Medical supply cupboards are known, however there appears to be no way for companies that make such medical supply cupboards available for people to use in their organisation to effectively monitor the use of the cupboard and/or its content in real time.

In a very basic form a medical supplies cupboard may merely be a cabinet having a plurality of shelves or drawers in which for example band aids, bandages, creams, syringes and other medical supplies are kept. Company employees can, at will, take items from such a cabinet or container. Consequently, it is often found in commercial premises that the state of a first aid cabinet or container leaves a lot to be desired. In order to maintain a more strict control over such cabinets some companies will restrict the number of users that can access the cabinet, but restricted access can be detrimental to the timely treatment of an injured or unwell person in an emergency situation.

A manual record of items coming out and going into the cabinet may also be kept in order to keep a track of stock levels. However, this is time consuming and not necessarily accurate as it is prone to human error. In certain emergencies when there is no time to complete the paper work, errors can creep into stock level documentation. This can result in items not being available when they are most needed potentially exposing companies to liability for lack of proper care.

Whilst some companies request their staff to inform a person responsible for maintaining the cabinet when certain items are running low, this often does not happen. This can cause problems when items are required in an emergency but stock has run out. When stock is running low it also takes time for the person responsible for the cabinet to re-order and supplement the cabinet as appropriate.

Some cabinets are subject to “unexplained stock losses”. Again, stock take on documentation in such instances will not be maintained and therefore can cause problems downstream. As a result a system for checking out medical supplies and medication is not ideal and is commonly subject to stock losses.

On some occasions it may be that the wrong items are taken from the cabinet to treat a particular injury, or that the cabinet user is unsure about what items to take or how to use them. The risk of injury complications can be increased where incorrect treatment is given, or where correct treatment is not given quickly.

### OBJECT OF THE INVENTION

It is therefore an object of the present invention to provide a medical supply cabinet or container which overcomes or at least partially ameliorates some of the abovementioned disadvantages or which at least provides the public with a useful choice.

Alternately, it is an object of the present invention to provide a method of monitoring the inventory of a medical supply cabinet or container which overcomes or at least partially ameliorates some of the abovementioned disadvantages or which at least provides the public with a useful choice.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

For the purpose of this specification, where method steps are described in sequence, the sequence does not necessarily mean that the steps are to be chronologically ordered in that sequence, unless there is no other logical manner of interpreting the sequence.

### SUMMARY OF THE INVENTION

In one aspect the invention can be said to broadly consist in a monitored dispensary comprising or including

a storage device to which multiple users have access, said storage device having a containment region inside of which items are stored, and

a monitoring system,

said monitoring system comprising at least

a) one or more detectors located inside the containment region and able to detect the removal and/or replacement and/or addition of items, and/or the weight of items prior to, during, and/or subsequent to any removal, replacement or addition transaction, and

b) one or more devices to obtain identity information derived from the user of the cabinet at the time that the transaction was effected,

Preferably said system further comprises a processor which is able to process the output of the sensors and the device in order to
i) create a record of each transaction, and
ii) associate the identity of the user with the transaction record, and
iii) monitor changes in the presence, absence and/or weight of items in the storage device from one transaction to the next.
   Preferably the system is also able report information generated by the functionalities of i), ii) and/or iii).
   Preferably the items contain or are items for use in medical treatment.
   Preferably said storage device is a cabinet.
   Preferably said storage device is wall hung, floor standing or portable.
   Preferably said storage device has a door or closure to enclose the containment region.
   Preferably said storage device is accessible only by a predetermined group of users.
   Preferably said storage device is freely accessible by any user, said user needing no prior authorisation or qualification to be able to access the storage device.
   Preferably said storage device has a fingerprint reader or scanner.
   Preferably said storage device is accessible only after a user has permitted their fingerprint to be read or scanned.
   Preferably said storage device has a cross shaped profile.
   Preferably said storage device is or can be illuminated.
   Preferably said containment region has one compartment or is divided into two or more compartments.
   Preferably each of said compartments is designated to receive either:
   a) items suitable for treating a particular type or injury or for providing treatment in response to a particular type of accident, and/or
   b) items falling into a particular category, said items having been categorised according to type, size, weight, use or some other physical characteristic.
   Preferably there are one or more bays inside a compartment, said bay or each of said bays designated to received either:
a) items suitable for treating a particular type or injury or for providing treatment in response to a particular type of accident, and/or
b) items falling into a particular category, said items having been categorised according to type, size, weight, use or some other physical characteristic.
   Preferably said storage device has or communicates with an input device which enables a user to specify a particular type of injury, wherein said input results in indicators inside the containment region indicating the location of items which may be useful in treating the specified type of injury.
   Preferably said indicators are LED lights.
   Preferably said device to obtain identity information derived from the user is a camera or optical device.
   Preferably said identity information derived from the user is a still image of the user, and preferably a still image of the face of the user.
   Preferably said identity information derived from the user is a video recording the user.
   Preferably said device to obtain identity information derived from the user is a card or tag reader.
   Preferably said identity information derived from the user is a unique identification code or signal.
   Preferably said device to obtain identity information derived from the user is a microphone or other audio recording device.
   Preferably said device to obtain identity information derived from the user is a finger print reader or scanner.
   Preferably said identity information derived from the user is a finger print image, scan or reading.
   Preferably the system is also able report information generated by the functionalities of i), ii) and/or iii).
   Preferably monitoring changes in the presence, absence and/or weight of items in the storage device from one transaction to the next results in:
   a) the reporting of stock levels of items contained inside the device, and/or
   b) the generating of notifications as items are taken, and/or as the stock levels of particular types or categories of item fall below a predetermined threshold level.
      Preferably there is real time reporting of stock levels of items contained inside the device.
      Preferably the monitoring system comprises an electronic data storage medium which can store transaction records and/or information generated by the functionalities of i), ii) and/or iii).
      Preferably reporting results in communication with one or more of:
   a) a computer, and
   b) a personal mobile device.
      Preferably components of the system are able to communicate information between them by way of wireless or cabled transmission.
      Preferably the system comprises at least one receiver and other necessary electronics to receive information transmitted via the internet, a local network or a cellular network.
      Preferably the system comprises a data input terminal which permits the entry of data to be processed by the system.
      Preferably the data input terminal permits manual data entry to the system.
      Preferably the system is able to interface with or refer to an independent source of time and/or date data.
      Preferably said system can process output from the sensors and/or the device for obtaining identity information in order to determine one or more of:
      a) what item(s) was/were taken,
      b) the number of such items taken,
      c) when the item(s) was/were taken, and
      d) what item(s) were added to the cabinet,
      e) the number of such items added,
      f) when the item(s) were added, and
      g) the cabinet being accessed but no items were taken or added.
      Preferably at least some of said sensors are load sensors.
      Preferably the items are arranged in a stacked formation.
      Preferably the detector is a load sensor located at the base of the stack.
      Preferably there is at least one sensor associated with a particular compartment or bay of the containment area to detect the presence, absence or change in weight of the item or items held in the compartment or bay.
      Preferably there is at least one sensor associated with each compartment or bay of the containment area to detect the presence, absence or change in weight of the item or items held in each of the compartments or bays.
      Preferably said items are containers which contain a further collection of items, said collection of items being
      a) suitable for treating a particular type or injury or for providing treatment in response to a particular type of accident, and/or
      b) a collection of items belonging to a particular category, said items having been categorised according to type, size, weight, use or some other physical characteristic.
      Preferably said items are standardised items.
      Preferably said items are grouped according to type, size, weight, use or some other physical characteristic.
      Preferably there is at least one sensor associated with a particular group of items to detect the presence, absence or change in weight of the items in that group.
      Preferably there is at least one sensor associated with a each group of items to detect the presence, absence or change in weight of the items in each of the groups.
      Preferably the items in a group of items are arranged in a stacked formation.
      Preferably the detector is a load sensor located at the base of the stack.
      Preferably the system may include or refer to a database containing pre-recorded information about one or more of the items or groups of items stocked in the cabinet.
      Preferably the system also has functionality for monitoring the expiry dates of items in the cabinet.
      Preferably the storage device has one or more scanners or readers located inside the containment region to scan or read expiry date information from the items.
      Preferably there are indicators located on or inside the storage device to provide information about the stock level and/or sterility of one or more of the items stocked in the cabinet.
      Preferably said indicators are visual or audible indicators which emit a display or signal corresponding to the stock level.
      Preferably said indicator(s) is/are located on the exterior of the storage device to indicate when at least some of the items inside the device are at a low stock level.
      Preferably said indicator(s) is/are located inside the containment region, each indicator being associated with a particular bay, compartment, item or group of items.
      Preferably said storage device has a screen.
      Preferably the screen is mounted within the containment region of the cabinet and/or on a door of the cabinet.
      Preferably said storage device has a speaker.
      Preferably said storage device has a means by which a user of the cabinet can indicate the type of injury for which treatment is desired.
      Preferably said means by which a user of the cabinet can indicate the type of injury for which treatment is desired is a manual input device.
      Preferably said manual input device is a keyboard.
      Preferably said manual input device is a series of buttons, toggles or switches, each in the series being designated as representing a particular type of injury.
      Preferably said cabinet comprises a plurality of indicators, each associated with a particular bay, compartment or location within the cabinet.
      Preferably said indicators are lights or LEDS.
      Preferably indication by the user that a particular type of injury is to be treated results in one or more of the following:
      a) activation of one or more indicators,
      b) the display of text, video or images on the screen, and
      c) the playing of audio through the speaker.
      Preferably said storage device has a communication system configured and adapted for communication with a remote station.
      Preferably said communication system comprises one or more information obtaining devices, one or more information delivering devices, one or more transmitters and one or more receivers.
      Preferably at least one transmitter is configured for transmitting the information obtained to the remote station.
      Preferably at least one receiver is configured for receiving information from the remote station.
      Preferably said information obtaining device comprises one or more of:
      a) a camera,
      b) a microphone, and
      c) a manual input device, preferably a keyboard.
      Preferably said information delivery device comprises one or more of:
      a) a screen, and
      b) a speaker.
      Preferably the communication system has a processor which may be used to process data before it is transmitted or after it is received.
      Preferably the communication system has associated software which may be used to process data before it is transmitted or after it is received.
      Preferably the communication system further comprises a portable device which can be used away from the cabinet.
      Preferably the portable device comprises at least an information obtaining device and an information delivery device.
      Preferably the portable device has a transmitter and/or a receiver configured to communicate with one or both of:
      a) said remote station, and
      b) at least one other component of the communication system.
      Preferably such communication is wireless.
      Preferably such communication is by way of an internet or network connection.
      Preferably said storage device has one or more sensors to detect when the portable device has been uplifted by a user.
      Preferably detecting that the portable device has been uplifted by a user results in the establishment of a connection for communication between said portable device and one or both of:
      a) said remote station, and
      b) at least one other component of the communication system.
      Preferably said communication system communicates with a data storage device.
      Preferably said communication system communicates with an electronic data storage medium.
      Preferably said communication system is integrated with or communicates with the monitoring system.
      Preferably said monitoring system is able to associate information processed by the communication system with a particular transaction record.

In another aspect the invention can be said to broadly consist in a storage device to which multiple users have access, said storage device having a containment region inside of which items are stored, and

one or more detectors located inside the containment region and able to detect the removal and/or replacement and/or addition of items, and/or the weight of items subsequent to any replacement or addition transaction

Preferably the storage device further comprises one or more devices to obtain identity information derived from the user of the cabinet at the time that the transaction was effected.
Preferably the storage device has one or more scanners or readers located inside the containment region to scan or read expiry date information from the items.
Preferably the items contain or are items for use in medical treatment.
Preferably said storage device is a cabinet.
Preferably said storage device is wall hung, floor standing or portable.
Preferably said storage device has a door or closure to enclose the containment region.
Preferably said storage device is accessible only by a predetermined group of users.
Preferably said storage device is freely accessible by any user, said user needing no prior authorisation or qualification to be able to access the storage device.
Preferably said storage device has a fingerprint reader or scanner.
Preferably said storage device is accessible only after a user has permitted their fingerprint to be read or scanned.
Preferably said storage device has a cross shaped profile.
Preferably said storage device is or can be illuminated.
Preferably said containment region has one compartment or is divided into two or more compartments.
Preferably each of said compartments is designated to receive either:
a) items suitable for treating a particular type or injury or for providing treatment in response to a particular type of accident, and/or
b) items falling into a particular category, said items having been categorised according to type, size, weight, use or some other physical characteristic.
Preferably there are one or more bays inside a compartment, said bay or each of said bays designated to received either:
a) items suitable for treating a particular type or injury or for providing treatment in response to a particular type of accident, and/or
b) items falling into a particular category, said items having been categorised according to type, size, weight, use or some other physical characteristic.
Preferably said storage device has or communicates with an input device which enables a user to specify a particular type of injury, wherein said input results in indicators inside the containment region indicating the location of items which may be useful in treating the specified type of injury.
Preferably said indicators are LED lights.
Preferably said device to obtain identity information derived from the user is a camera or optical device.
Preferably said identity information derived from the user is a still image of the user, and preferably a still image of the face of the user.
Preferably said identity information derived from the user is a video recording the user.
Preferably said device to obtain identity information derived from the user is a card or tag reader.
Preferably said identity information derived from the user is a unique identification code or signal.
Preferably said device to obtain identity information derived from the user is a microphone or other audio recording device.
Preferably said device to obtain identity information derived from the user is a finger print reader or scanner.
Preferably said identity information derived from the user is a finger print image, scan or reading.
Preferably at least some of said sensors are load sensors.
Preferably the items are arranged in a stacked formation.
Preferably the detector is a load sensor located at the base of the stack.
Preferably there is at least one sensor associated with a particular compartment or bay of the containment area to detect the presence, absence or change in weight of the item or items held in the compartment or bay.
Preferably there is at least one sensor associated with each compartment or bay of the containment area to detect the presence, absence or change in weight of the item or items held in each of the compartments or bays.
Preferably said items are containers which contain a further collection of items, said collection of items being
a) suitable for treating a particular type or injury or for providing treatment in response to a particular type of accident, and/or
b) a collection of items belonging to a particular category, said items having been categorised according to type, size, weight, use or some other physical characteristic.
Preferably said items are standardised items.
Preferably said items are grouped according to type, size, weight, use or some other physical characteristic.
Preferably there is at least one sensor associated with a particular group of items to detect the presence, absence or change in weight of the items in that group.
Preferably there is at least one sensor associated with a each group of items to detect the presence, absence or change in weight of the items in each of the groups.
Preferably the items in a group of items are arranged in a stacked formation.
Preferably the detector is a load sensor located at the base of the stack.
Preferably the system may include or refer to a database containing pre-recorded information about one or more of the items or groups of items stocked in the cabinet.
Preferably there are indicators located on or inside the storage device to provide information about the stock level and/or sterility of one or more of the items stocked in the cabinet.
Preferably said indicators are visual or audible indicators which emit a display or signal corresponding to the stock level.
Preferably said indicator(s) is/are located on the exterior of the storage device to indicate when at least some of the items inside the device are at a low stock level.
Preferably said indicator(s) is/are located inside the containment region, each indicator being associated with a particular bay, compartment, item or group of items.
Preferably said storage device has a screen.
Preferably the screen is mounted within the containment region of the cabinet and/or on a door of the cabinet.
Preferably said storage device has a speaker.
Preferably said storage device has a means by which a user of the cabinet can indicate the type of injury for which treatment is desired.
Preferably said means by which a user of the cabinet can indicate the type of injury for which treatment is desired is a manual input device.
Preferably said manual input device is a keyboard.
Preferably said manual input device is a series of buttons, toggles or switches, each in the series being designated as representing a particular type of injury.
Preferably said cabinet comprises a plurality of indicators, each associated with a particular bay, compartment or location within the cabinet.
Preferably said indicators are lights or LEDS.
Preferably indication by the user that a particular type of injury is to be treated results in one or more of the following:
a) activation of one or more indicators,
b) the display of text, video or images on the screen, and
c) the playing of audio through the speaker.
Preferably said storage device has a communication system configured and adapted for communication with a remote station.
Preferably said communication system comprises one or more information obtaining devices, one or more information delivering devices, one or more transmitters and one or more receivers.
Preferably at least one transmitter is configured for transmitting the information obtained to the remote station.
Preferably at least one receiver is configured for receiving information from the remote station.
Preferably said information obtaining device comprises one or more of:
a) a camera,
b) a microphone, and
c) a manual input device, preferably a keyboard.
Preferably said information delivery device comprises one or more of:
a) a screen, and
b) a speaker.
Preferably the communication system has a processor which may be used to process data before it is transmitted or after it is received.
Preferably the communication system has associated software which may be used to process data before it is transmitted or after it is received.
Preferably the communication system further comprises a portable device which can be used away from the cabinet.
Preferably the portable device comprises at least an information obtaining device and an information delivery device.
Preferably the portable device has a transmitter and/or a receiver configured to communicate with one or both of:
a) said remote station, and
b) at least one other component of the communication system.
Preferably such communication is wireless.
Preferably such communication is by way of an internet or network connection.
Preferably said storage device has one or more sensors to detect when the portable device has been uplifted by a user.
Preferably detecting that the portable device has been uplifted by a user results in the establishment of a connection for communication between said portable device and one or both of:
a) said remote station, and
b) at least one other component of the communication system.
Preferably said communication system communicates with a data storage device.
Preferably said communication system communicates with an electronic data storage medium.
Preferably said communication system is integrated with or communicates with the monitoring system.
Preferably said monitoring system is able to associate information processed by the communication system with a particular transaction record.

In another aspect the invention can be said to broadly consist in a monitored dispensary comprising or including

a storage device to which multiple users are to have access to remove standardised items, and

sensors, as part of a monitoring system, positioned to reveal the presence or absence of standardised items stored by the storage device,

a user identification functionality as part of the monitoring system, and

a report and/or reordering functionality as part of the monitoring system responsive to each sensor and able to tie any sensed item removal to a user;

wherein at least some of the sensors are or include a load cell or the like whereby a standardised item from which some content can be removed is sensed when replaced as having had some of its content removed.

Preferably the system is also able output information generated by the user identification, reporting and/or reordering functionalities.
Preferably the items contain or are items for use in medical treatment.
Preferably said storage device is a cabinet.
Preferably said storage device is wall hung, floor standing or portable.
Preferably said storage device has a door or closure to enclose the containment region.
Preferably said storage device is accessible only by a predetermined group of users.
Preferably said storage device is freely accessible by any user, said user needing no prior authorisation or qualification to be able to access the storage device.
Preferably said storage device has a fingerprint reader or scanner.
Preferably said storage device is accessible only after a user has permitted their fingerprint to be read or scanned.
Preferably said storage device has a cross shaped profile.
Preferably said storage device is or can be illuminated.
Preferably said storage device has one compartment or is divided into two or more compartments.
Preferably each of said compartments is designated to receive either:
a) items suitable for treating a particular type or injury or for providing treatment in response to a particular type of accident, and/or
b) items falling into a particular category, said items having been categorised according to type, size, weight, use or some other physical characteristic.
Preferably there are one or more bays inside a compartment, said bay or each of said bays designated to received either:
a) items suitable for treating a particular type or injury or for providing treatment in response to a particular type of accident, and/or
b) items falling into a particular category, said items having been categorised according to type, size, weight, use or some other physical characteristic.
   Preferably said storage device has or communicates with an input device which enables a user to specify a particular type of injury, wherein said input results in indicators inside the containment region indicating the location of items which may be useful in treating the specified type of injury.
   Preferably said indicators are LED lights.
   Preferably said user identification functionality involves the operative use of a device to obtain identity information derived from a user of the cabinet.
   Preferably device to obtain identity information derived from the user is a camera or optical device.
   Preferably said identity information derived from the user is a still image of the user, and preferably a still image of the face of the user.
   Preferably said identity information derived from the user is a video recording the user.
   Preferably said device to obtain identity information derived from the user is a card or tag reader.
   Preferably said identity information derived from the user is a unique identification code or signal.
   Preferably said device to obtain identity information derived from the user is a microphone or other audio recording device.
   Preferably said device to obtain identity information derived from the user is a finger print reader or scanner.
   Preferably said identity information derived from the user is a finger print image, scan or reading.
   Preferably the report and/or re-order functionality results in:
   a) the reporting of stock levels of items contained inside the storage device, and/or
   b) the generating of notifications as items are taken, and/or as the stock levels of particular types or categories of item fall below a predetermined threshold level.
      Preferably there is real time reporting of stock levels of items contained inside the device.
      Preferably the monitoring system comprises an electronic data storage medium which can store transaction records and/or information generated by user identification functionality and report and/or reordering functionality.
      Preferably reporting results in communication with one or more of:
   a) a computer, and
   b) a personal mobile device.
      Preferably components of the system are able to communicate information between them by way of wireless or cabled transmission.
      Preferably the system comprises at least one receiver and other necessary electronics to receive information transmitted via the internet, a local network or a cellular network.
      Preferably the system comprises a data input terminal which permits the entry of data to be processed by the system.
      Preferably the data input terminal permits manual data entry to the system.
      Preferably the system is able to interface with or refer to an independent source of time and/or date data.
      Preferably said system can process output from the sensors and/or the device for obtaining identity information in order to determine one or more of:
      a) what item(s) was/were taken,
      b) the number of such items taken,
      c) when the item(s) was/were taken, and
      d) what item(s) were added to the cabinet,
      e) the number of such items added,
      f) when the item(s) were added, and
      g) the cabinet being accessed but no items were taken or added.
      Preferably at least some of said sensors are load sensors.
      Preferably the items are arranged in a stacked formation.
      Preferably the detector is a load sensor located at the base of the stack.
      Preferably there is at least one sensor associated with a particular compartment or bay of the storage device to detect the presence, absence or change in weight of the item or items held in the compartment or bay.
      Preferably there is at least one sensor associated with each compartment or bay of the storage device to detect the presence, absence or change in weight of the item or items held in each of the compartments or bays.
      Preferably said items are containers which contain a further collection of items, said collection of items being
      a) suitable for treating a particular type or injury or for providing treatment in response to a particular type of accident, and/or
      b) a collection of items belonging to a particular category, said items having been categorised according to type, size, weight, use or some other physical characteristic.
      Preferably said items are standardised items.
      Preferably said items are grouped according to type, size, weight, use or some other physical characteristic.
      Preferably there is at least one sensor associated with a particular group of items to detect the presence, absence or change in weight of the items in that group.
      Preferably there is at least one sensor associated with a each group of items to detect the presence, absence or change in weight of the items in each of the groups.
      Preferably the items in a group of items are arranged in a stacked formation.
      Preferably the detector is a load sensor located at the base of the stack.
      Preferably the system may include or refer to a database containing pre-recorded information about one or more of the items or groups of items stocked in the cabinet.
      Preferably the system also has functionality for monitoring the expiry dates of items in the cabinet.
      Preferably the storage device has one or more scanners or readers located inside the containment region to scan or read expiry date information from the items.
      Preferably there are indicators located on or inside the storage device to provide information about the stock level and/or sterility of one or more of the items stocked in the cabinet.
      Preferably said indicators are visual or audible indicators which emit a display or signal corresponding to the stock level.
      Preferably said indicator(s) is/are located on the exterior of the storage device to indicate when at least some of the items inside the device are at a low stock level.
      Preferably said indicator(s) is/are located inside the storage device, each indicator being associated with a particular bay, compartment, item or group of items.
      Preferably said storage device has a screen.
      Preferably the screen is mounted within the containment region of the cabinet and/or on a door of the cabinet.
      Preferably said storage device has a speaker.
      Preferably said storage device has a means by which a user of the cabinet can indicate the type of injury for which treatment is desired.
      Preferably said means by which a user of the cabinet can indicate the type of injury for which treatment is desired is a manual input device.
      Preferably said manual input device is a keyboard.
      Preferably said manual input device is a series of buttons, toggles or switches, each in the series being designated as representing a particular type of injury.
      Preferably said cabinet comprises a plurality of indicators, each associated with a particular bay, compartment or location within the cabinet.
      Preferably said indicators are lights or LEDS.
      Preferably indication by the user that a particular type of injury is to be treated results in one or more of the following:
      a) activation of one or more indicators,
      b) the display of text, video or images on the screen, and
      c) the playing of audio through the speaker.
      Preferably said storage device has a communication system configured and adapted for communication with a remote station.
      Preferably said communication system comprises one or more information obtaining devices, one or more information delivering devices, one or more transmitters and one or more receivers.
      Preferably at least one transmitter is configured for transmitting the information obtained to the remote station.
      Preferably at least one receiver is configured for receiving information from the remote station.
      Preferably said information obtaining device comprises one or more of:
      a) a camera,
      b) a microphone, and
      c) a manual input device, preferably a keyboard.
      Preferably said information delivery device comprises one or more of:
      a) a screen, and
      b) a speaker.
      Preferably the communication system has a processor which may be used to process data before it is transmitted or after it is received.
      Preferably the communication system has associated software which may be used to process data before it is transmitted or after it is received.
      Preferably the communication system further comprises a portable device which can be used away from the cabinet.
      Preferably the portable device comprises at least an information obtaining device and an information delivery device.
      Preferably the portable device has a transmitter and/or a receiver configured to communicate with one or both of:
      a) said remote station, and
      b) at least one other component of the communication system.
      Preferably such communication is wireless.
      Preferably such communication is by way of an internet or network connection.
      Preferably said storage device has one or more sensors to detect when the portable device has been uplifted by a user.
      Preferably detecting that the portable device has been uplifted by a user results in the establishment of a connection for communication between said portable device and one or both of:
      a) said remote station, and
      b) at least one other component of the communication system.
      Preferably said communication system communicates with a data storage device.
      Preferably said communication system communicates with an electronic data storage medium.
      Preferably said communication system is integrated with or communicates with the monitoring system.
      Preferably said monitoring system is able to associate information processed by the communication system with a particular transaction record.

In another aspect the invention can be said to broadly consist in an access and storage system to allow for multiple user access, the system comprising or including

a storage device to allow access to standardised items of different classes to be stored therein, and

as part of a monitoring system, a stored inventory maintenance sensor system and a user identification system;

wherein the stored inventory maintenance sensor system is for standardised items of different classes such that each item of a class will have its removal sensed, any restocking sensed and any replacement of a removed item, but at a lesser weight, sensed;

and wherein the user identification system can tie a user to the time and class of each item removed, and the weight of any item replaced at a lesser weight;

and wherein the stored inventory maintenance sensor system and user identification system feed as inputs of the monitoring system

Preferably the monitoring system is also able output information generated by the user identification system and/or stored inventory maintenance sensor system.
Preferably the items contain or are items for use in medical treatment.
Preferably said storage device is a cabinet.
Preferably said storage device is wall hung, floor standing or portable.
Preferably said storage device has a door or closure to enclose the containment region.
Preferably said storage device is accessible only by a predetermined group of users.
Preferably said storage device is freely accessible by any user, said user needing no prior authorisation or qualification to be able to access the storage device.
Preferably said storage device has a fingerprint reader or scanner.
Preferably said storage device is accessible only after a user has permitted their fingerprint to be read or scanned.
Preferably said storage device has a cross shaped profile.
Preferably said storage device is or can be illuminated.
Preferably said storage device has one compartment or is divided into two or more compartments.
Preferably each of said compartments is designated to receive either:
a) items suitable for treating a particular type or injury or for providing treatment in response to a particular type of accident, and/or
b) items falling into a particular category, said items having been categorised according to type, size, weight, use or some other physical characteristic.
Preferably there are one or more bays inside a compartment, said bay or each of said bays designated to received either:
a) items suitable for treating a particular type or injury or for providing treatment in response to a particular type of accident, and/or
b) items falling into a particular category, said items having been categorised according to type, size, weight, use or some other physical characteristic.
   Preferably said storage device has or communicates with an input device which enables a user to specify a particular type of injury, wherein said input results in indicators inside the containment region indicating the location of items which may be useful in treating the specified type of injury.
   Preferably said indicators are LED lights.
   Preferably said user identification system involves the operative use of a device to obtain identity information derived from a user of the cabinet.
   Preferably device to obtain identity information derived from the user is a camera or optical device.
   Preferably said identity information derived from the user is a still image of the user, and preferably a still image of the face of the user.
   Preferably said identity information derived from the user is a video recording the user.
   Preferably said device to obtain identity information derived from the user is a card or tag reader.
   Preferably said identity information derived from the user is a unique identification code or signal.
   Preferably said device to obtain identity information derived from the user is a microphone or other audio recording device.
   Preferably said device to obtain identity information derived from the user is a finger print reader or scanner.
   Preferably said identity information derived from the user is a finger print image, scan or reading.
   Preferably the monitoring system is also able report information generated by the user identification system and the stored inventory maintenance sensor system.
   Preferably the monitoring system is able to:
   a) report the stock levels of items contained inside the storage device, and/or
   b) generate notifications as items are taken, and/or as the stock levels of particular types or categories of item fall below a predetermined threshold level.
      Preferably the monitoring system comprises an electronic data storage medium which can store transaction records and/or information generated by the user identification system and the stored inventory maintenance sensor system.
      Preferably the reporting of stock levels of items contained inside the storage device is in real time.
      Preferably reporting results in communication with one or more of:
   a) a computer, and
   b) a personal mobile device.
      Preferably components of the systems are able to communicate information between them by way of wireless or cabled transmission.
      Preferably at least one of the systems comprises at least one receiver and other necessary electronics to receive information transmitted via the internet, a local network or a cellular network.
      Preferably at least one of the systems comprises a data input terminal which permits the entry of data to be processed by the system.
      Preferably the data input terminal permits manual data entry to the system.
      Preferably at least one of the systems is able to interface with or refer to an independent source of time and/or date data.
      Preferably said monitoring system can process output from the sensors and/or the device for obtaining identity information in order to determine one or more of:
      a) what item(s) was/were taken,
      b) the number of such items taken,
      c) when the item(s) was/were taken, and
      d) what item(s) were added to the cabinet,
      e) the number of such items added,
      f) when the item(s) were added, and
      g) the cabinet being accessed but no items were taken or added.
      Preferably at least some of said sensors are load sensors.
      Preferably the items are arranged in a stacked formation.
      Preferably the detector is a load sensor located at the base of the stack.
      Preferably there is at least one sensor associated with a particular compartment or bay of the storage device to detect the presence, absence or change in weight of the item or items held in the compartment or bay.
      Preferably there is at least one sensor associated with each compartment or bay of the storage device to detect the presence, absence or change in weight of the item or items held in each of the compartments or bays.
      Preferably said items are containers which contain a further collection of items, said collection of items being
      a) suitable for treating a particular type or injury or for providing treatment in response to a particular type of accident, and/or
      b) a collection of items belonging to a particular category, said items having been categorised according to type, size, weight, use or some other physical characteristic.
      Preferably said items are standardised items.
      Preferably said items are grouped according to type, size, weight, use or some other physical characteristic.
      Preferably there is at least one sensor associated with a particular group of items to detect the presence, absence or change in weight of the items in that group.
      Preferably there is at least one sensor associated with a each group of items to detect the presence, absence or change in weight of the items in each of the groups.
      Preferably the items in a group of items are arranged in a stacked formation.
      Preferably the detector is a load sensor located at the base of the stack.
      Preferably the system may include or refer to a database containing pre-recorded information about one or more of the items or groups of items stocked in the cabinet.
      Preferably at least one of the systems also has functionality for monitoring the expiry dates of items in the cabinet.
      Preferably the storage device has one or more scanners or readers located inside the containment region to scan or read expiry date information from the items.
      Preferably there are indicators located on or inside the storage device to provide information about the stock level and/or sterility of one or more of the items stocked in the cabinet.
      Preferably said indicators are visual or audible indicators which emit a display or signal corresponding to the stock level.
      Preferably said indicator(s) is/are located on the exterior of the storage device to indicate when at least some of the items inside the device are at a low stock level.
      Preferably said indicator(s) is/are located inside the storage device, each indicator being associated with a particular bay, compartment, item or group of items.
      Preferably said storage device has a screen.
      Preferably the screen is mounted within the containment region of the cabinet and/or on a door of the cabinet.
      Preferably said storage device has a speaker.
      Preferably said storage device has a means by which a user of the cabinet can indicate the type of injury for which treatment is desired.
      Preferably said means by which a user of the cabinet can indicate the type of injury for which treatment is desired is a manual input device.
      Preferably said manual input device is a keyboard.
      Preferably said manual input device is a series of buttons, toggles or switches, each in the series being designated as representing a particular type of injury.
      Preferably said cabinet comprises a plurality of indicators, each associated with a particular bay, compartment or location within the cabinet.
      Preferably said indicators are lights or LEDS.
      Preferably indication by the user that a particular type of injury is to be treated results in one or more of the following:
      a) activation of one or more indicators,
      b) the display of text, video or images on the screen, and
      c) the playing of audio through the speaker.
      Preferably said storage device has a communication system configured and adapted for communication with a remote station.
      Preferably said communication system comprises one or more information obtaining devices, one or more information delivering devices, one or more transmitters and one or more receivers.
      Preferably at least one transmitter is configured for transmitting the information obtained to the remote station.
      Preferably at least one receiver is configured for receiving information from the remote station.
      Preferably said information obtaining device comprises one or more of:
      a) a camera,
      b) a microphone, and
      c) a manual input device, preferably a keyboard.
      Preferably said information delivery device comprises one or more of:
      a) a screen, and
      b) a speaker.
      Preferably the communication system has a processor which may be used to process data before it is transmitted or after it is received.
      Preferably the communication system has associated software which may be used to process data before it is transmitted or after it is received.
      Preferably the communication system further comprises a portable device which can be used away from the cabinet.
      Preferably the portable device comprises at least an information obtaining device and an information delivery device.
      Preferably the portable device has a transmitter and/or a receiver configured to communicate with one or both of:
      a) said remote station, and
      b) at least one other component of the communication system.
      Preferably such communication is wireless.
      Preferably such communication is by way of an internet or network connection.
      Preferably said storage device has one or more sensors to detect when the portable device has been uplifted by a user.
      Preferably detecting that the portable device has been uplifted by a user results in the establishment of a connection for communication between said portable device and one or both of:
      a) said remote station, and
      b) at least one other component of the communication system.
      Preferably said communication system communicates with a data storage device.
      Preferably said communication system communicates with an electronic data storage medium.
      Preferably said communication system is integrated with or communicates with the monitoring system.
      Preferably said monitoring system is able to associate information processed by the communication system with a particular transaction record.

In another aspect the invention can be said to broadly consist in a communication system for use with a storage device for medical supplies, said communication system configured and adapted for communication with a station remote from the storage device, and comprising one or more information obtaining devices, one or more information delivering devices, one or more transmitters and one or more receivers.

Preferably at least one transmitter is configured for transmitting the information obtained to the remote station.
Preferably at least one receiver is configured for receiving information from the remote station.
Preferably said information obtaining device comprises one or more of:
a) a camera,
b) a microphone, and
c) a manual input device, preferably a keyboard.
Preferably said information delivery device comprises one or more of:
a) a screen, and
b) a speaker.
Preferably the communication system has a processor which may be used to process data before it is transmitted or after it is received.
Preferably the communication system has associated software which may be used to process data before it is transmitted or after it is received.
Preferably the communication system comprises a portable device which can be used away from the storage device.
Preferably the portable device comprises at least an information obtaining device and an information delivery device.
Preferably the portable device has a transmitter and/or a receiver configured to communicate with one or both of:
a) said remote station, and
b) at least one other component of the communication system.
Preferably such communication is wireless.
Preferably such communication is by way of an internet or network connection.
Preferably said storage device has one or more sensors to detect when the portable device has been uplifted by a user.
Preferably detecting that the portable device has been uplifted by a user results in the establishment of a connection for communication between said portable device and one or both of:
a) said remote station, and
b) at least one other component of the communication system.
Preferably said communication system communicates with a data storage device.
Preferably said communication system communicates with an electronic data storage medium.
Preferably said communication system is integrated with or communicates with the monitoring system.
Preferably said monitoring system is able to associate information processed by the communication system with a particular transaction record.

In another aspect the invention may be said to broadly consist in a monitored dispensary as herein described with reference to the accompanying drawings.

In another aspect the invention may be said to broadly consist in a storage device as herein described with reference to the accompanying drawings.

In another aspect the invention may be said to broadly consist in an access and storage system to allow for multiple user access, as herein described with reference to the accompanying drawings.

In another aspect the invention can be said to broadly consist in a communication system for use with a storage device for medical supplies as herein described with reference to the accompanying drawings.

Other aspects of the invention may become apparent from the following description which is given by way of example only and with reference to the accompanying drawings.

As used herein the term “and/or” means “and” or “or”, or both.

As used herein “(s)” following a noun means the plural and/or singular forms of the noun.

The term “comprising” as used in this specification means “consisting at least in part of”. When interpreting statements in this specification [and claims] which include that term, the features, prefaced by that term in each statement, all need to be present but other features can also be present. Related terms such as “comprise” and “comprised” are to be interpreted in the same manner.

The entire disclosures of all applications, patents and publications, cited above and below, if any, are hereby incorporated by reference.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.)

### DESCRIPTION OF THE DRAWINGS

The invention will now be described by way of example only and with reference to the drawings in which:

FIG. 1: shows a medical supply cabinet of the present invention,

FIG. 1A: shows a medical supply cabinet of the present invention where the camera is obscured by the closed doors of the camera,

FIG. 2: shows a containment area inside of a housing of the medical supply cabinet of FIG. 1,

FIG. 3: shows the containment area of FIG. 2 holding a selection of medical supply items,

FIG. 4: shows a system for the monitoring, recording and reporting of information relating to stock levels inside of a medical supply cabinet of the present invention,

FIG. 5: shows an embodiment of a medical supply cabinet of the present invention which includes a display screen and visual indicators for items in the cabinet,

FIG. 6: shows an embodiment of a medical supply cabinet of the present invention which includes a communication system for communicating with a remote station, and

FIG. 7: shows an embodiment of a medical supply cabinet of the present invention which includes a data obtaining device to be worn by a user at an injury site, wherein the user worn device can communicate with a communication system of the cabinet such as that shown in FIG. 6.

### DETAILED DESCRIPTION OF THE INVENTION

The invention comprises a medical supplies cabinet 1 and a related system for stock control.

The cabinet 1 may be wall hung or floor standing, or in some embodiments may be portable. The cabinet 1 has a containment region 2 that is able to be enclosed. It is preferably able to be closed by a door or doors 3 that slide or hinge relative a cabinet housing 4. Alternatively the containment region 2 remains open at all times.

In some embodiments the doors 3 or other means of enclosing the containment region 2 are not secured, so that any person is able to access the contents of the cabinet 1. In other embodiments some form of lock or other form of security may be provided so that only certain people can access the cabinet 1. For example a swipe card may be read to actuate unlocking of the cabinet 1.

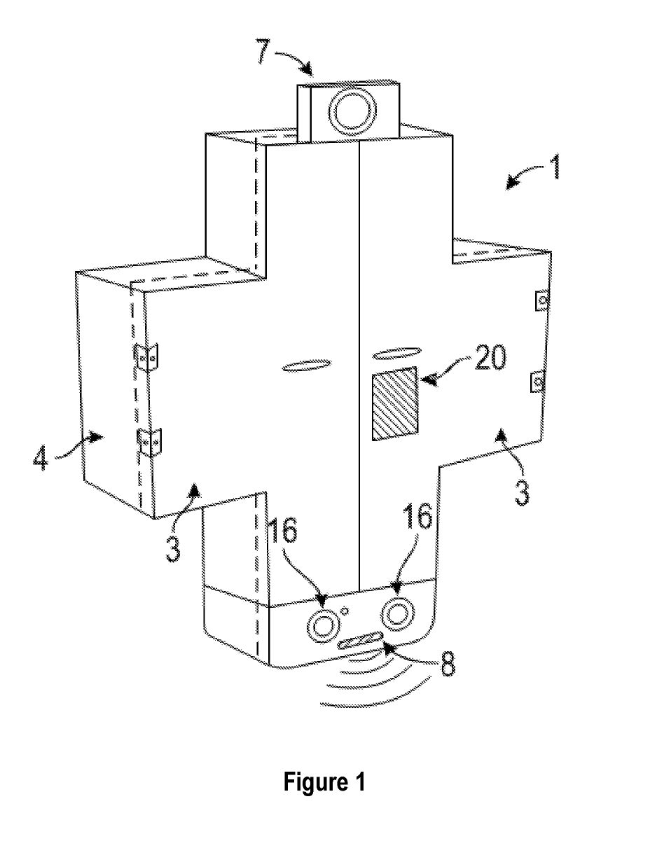

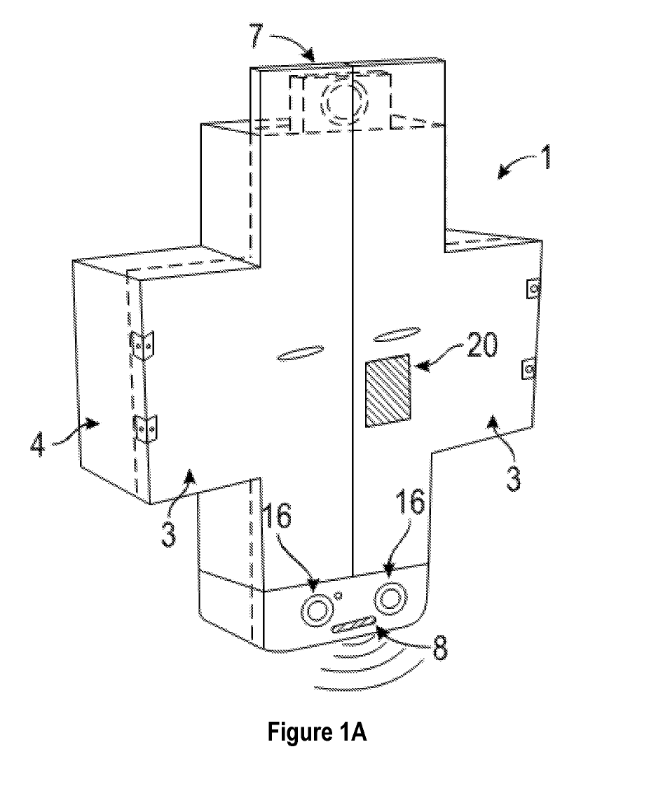

In some embodiments there may be a finger print scanner or reader 20 which is able to obtain and/or interpret information derived from a fingerprint of the user of the cabinet 1. Preferably the finger print scanner or reader 20 is positioned on the exterior of the cabinet 1, such that a person must permit their fingerprint to be scanned/read prior to being granted access to the interior of the cabinet 1. The information obtained by the scanner or reader 20 may be used to verify that the user is authorised to access the cabinet 1. The information obtained by the scanner or reader 20 may also, or alternatively, be stored by an electronic recording system. For example, the fingerprint may be as a record of the identity of the user of the cabinet for a particular transaction. In preferred embodiments the cabinet 1 has a distinctive cross shaped profile as shown in FIG. 1, so that in an emergency any person would likely recognise it as being a container for medical supplies. The cabinet 1 may be green in colour, or backlit with a green illumination, or otherwise illuminated. The illumination be constant or may pulse. For example, the cabinet may have a UPS, such as a battery or onboard generator, so that if there is a power outage during a natural disaster the cabinet can remain illuminated, and the illumination may pulse to attract the attention of those who are searching for medical supplies.

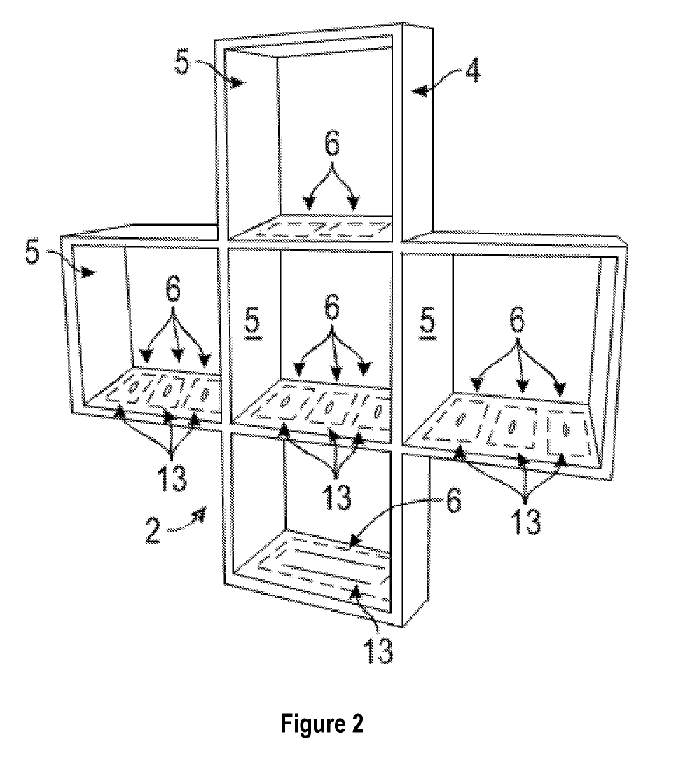

The containment region 2 may have one compartment or may be divided into two or more compartments 5 as shown in FIG. 2, such that medical supplies can be arranged in an organised manner. In some embodiments the, or each, compartment 5 may contain those supplies necessary to treat a specific type of injury or accident, or provide a specific type of treatment. For example there may be a compartment containing items suitable for treating burns and another compartment containing items suitable for treating cuts.

Within one or more of the compartments there may be one or more stock item bays 6 for receiving the various items to be held in the cabinet 1. The compartments 5, and the stock item bays 6 inside each compartment 5, may be reconfigurable in their dimensions and locations so that the layout of the containment region 2 can be customised for different applications. The stock item bays 6 may be designated to receive a specific type of stock item, for example a left hand bay may be designated to receive bandages, while an adjoining bay may be designated to receive sticking plasters.

In some embodiments there may be one or more indicators, each associated with a particular bay 6 inside the cabinet 1 (each of said bays being designated to receive a specific type of stock item), and preferably positioned proximate to the associated bay. There may be an input means for a user of the cabinet 1 to communicate the type of injury that they wish to treat. As a result of the input, the indicators will signal the location of those bays 6 that contain items which may be useful in treating the injury so that the needed items can rapidly be gathered by the user. For example, there may be an LED light positioned above each bay 6 inside the containment area 2. There may be a series of press buttons located on the inside or outside of the housing 4, each corresponding to a particular injury type, e.g. burns, cuts, eye injuries, CPR and trauma. If a user presses the button corresponding to a cut injury, the LED lights above those bays which hold antiseptic solution and butterfly strips may become illuminated to assist the user in locating items useful for treating the cut.

Preferably associated with the cabinet 1 is a camera 7. This camera 7 will record an image or footage of the person taking medical supplies from the cabinet 1. The image or footage may be time or date stamped and/or logged in an electronic recording system. In some embodiments time and/or date stamping occurs with reference to an independent source of time and/or date data to ensure the accuracy of the time and date records.

The camera 7 (or other optical device) may take a photo of the person using the cabinet 1, and preferably has suitable optics for close up imagery of the person using the cabinet 1. For example, the camera 7 may obtain a clear picture of the face of the user from which the user can be identified. For this reason it may be desirable that the camera 7 is located or positioned to obtain an image of the head and/or face region of a user as the user looks into the containment region 2 to view items. At this point the user will have had to come close enough to the cabinet 1 to reach and open the cabinet door 3, and the user's face will likely be positioned to give a line of sight into the containment region 2.

This photo or footage may be recorded automatically. This may be achieved by a proximity sensor 8 that triggers the recording, by a switch associated with the cabinet doors contact sensor (not shown) or other means of triggering a recording. For example, it may be desirable that the opening of the cabinet door triggers a photograph or video being taken. There may be a light or sound indicator to encourage the person to look toward the camera 7 as the photo is taken.

Alternatively the camera 7 may be set to continuously record, such as if it's a video camera or by way of time lapse stills. It may include a function to tag or take a photo as and when someone is engaging with the cabinet 1. For example, this may be triggered by the opening of the cabinet door 3.

In some embodiments the user may be directed to submit identity information when interacting with the cabinet 1, for example by swiping an identification card upon accessing the cabinet and then subsequently swiping a barcode on an item removed from the cabinet. However in some embodiments may be desirable that at least some of the user identity information is obtained passively or automatically, without relying on the user to take action (this could be instead of, or in addition to identity information volunteered by the user). For example, an image of the user may be recorded each and every time that the cabinet is accessed (for example, triggered by the opening door 3 of the cabinet) and/or regardless of whether any items are taken from or added to the containment region 2.

The camera 7, or other optical device, may be mounted externally of the cabinet 1 as shown in FIG. 1. However in some embodiments there may be means to hide or protect the camera so as to discourage and/or prevent users from tampering, in an effort to prevent their identity from being recorded. For example, as shown in FIG. 1A the doors 3 of the cabinet may extend upwardly to obscure the camera 7 from the user's view until the doors 3 have been opened. This may also serve to prevent the user from positioning anything in front of the camera lens prior to opening the doors to prevent themselves from being recorded.

It will be appreciated that other means of obscuring or shielding the camera 7 could be employed. As a further example, the camera could be encased or housed internally of the cabinet housing 4 itself where it is hidden and cannot be accessed by a user. A user accessing the cabinet may cause the camera 7 to emerge to the exterior of the cabinet housing 4 and obtain a photograph or video recording of the user, before retreating again to the interior of the cabinet housing 4.

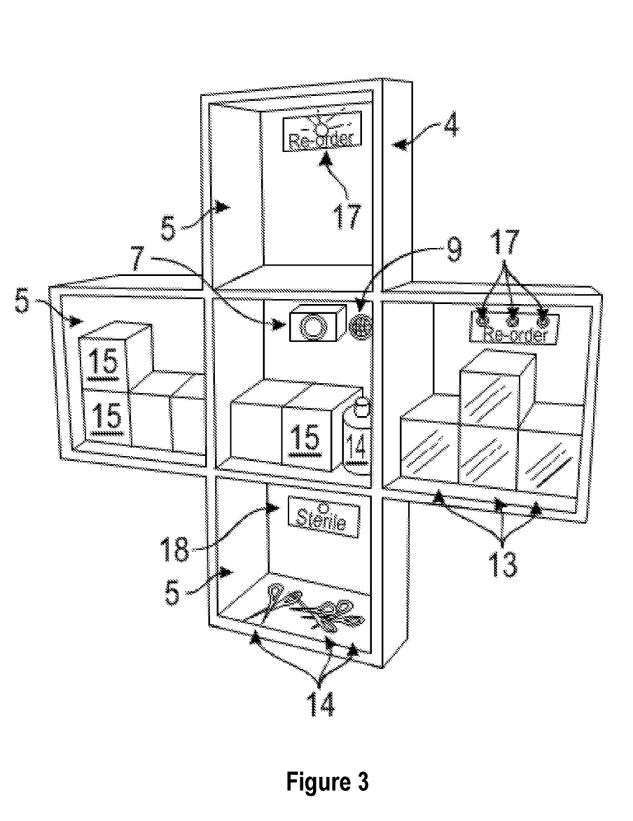

As a further alternative, the camera 7 it may be housed internally of the containment region 7 as shown in FIG. 3, such that the camera 7 cannot be accessed by a user for tampering without the user having first opened the cabinet doors 3 and looked into the containment region 2. By which time the camera 7 may have already recorded an image of the user (e.g. the users face) from which the user can be identified. In some embodiments a microphone 9 is provided inside or near the cabinet 1 to enable the recording of audio streams at the time that the cabinet 1 is accessed. The audio information made be date or time stamped and/or stored along with the visual information from the camera 7.

The supplies cabinet 1 includes or is associated with a system for recording activity of the cabinet, including transactions where users remove items from the cabinet and/or replace items back in the cabinet. In particular the system may be used for recording and/or monitoring stock levels in the cabinet 1. The system may also be used for reporting stock levels for the purposes of reordering stock. This reporting may cause automatic reordering of stock as stock is taken, or as stock levels fall below a certain threshold. Preferably the system is also able to detect and record transactions where new stock is added to the cabinet 1.

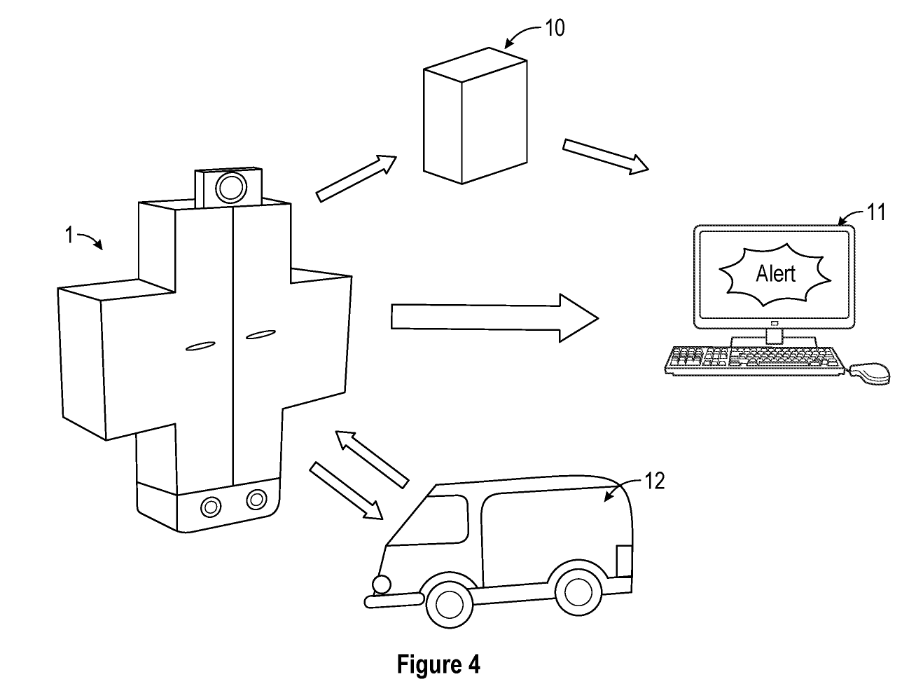

A schematic of an exemplary system is shown in FIG. 4. The output from sensors inside the cabinet 1 can be processed to yield information about stock levels inside the cabinet 1. The information processed by the system can be recorded in a data storage medium 10 to create a historical log of the activity of the cabinet 1. As an example, this can be referred to at a later date for health and safety auditing purposes. In health and safety recording, reporting and auditing applications it can be particularly useful to have a complete record of all transactions with the cabinet, the user identity for each of those transactions, and an indication of any items accessed during the transaction.

The information processed by the system can also be sent directly or indirectly to a computer 11 or mobile phone, such as that of a third party. For example, the system can create an incidence report of each transaction which is sent to the computer 11 of the person who is in charge of monitoring the stock levels and use of the cabinet. As an alternative example, the system may have an associated mobile phone application which enables a mobile phone user to simultaneously monitor the stock levels and use in relation to a number of supply cabinets for which that person is responsible. Furthermore the system may output information directly to a supplier or distributor 12, so that re-ordering and replenishment of stock items can be carried out. Preferably the re-ordering occurs automatically at by, or at the prompt of, the system.

In some embodiments, information processed by the system may be communicated via a Wi-Fi connection to a local network, or communicated as an SMS over a cellular or mobile network. Internet and/or network connections may also be used to input information into the system and effect system software updates.

In some embodiments the system comprises or communicates with a data storage device such as a network, hard drive or “the cloud” to create a log of transactions involving the cabinet and/or the items held in the cabinet. Preferably the log is electronically accessible. For example, a stock distributor or supplier may be able to input cabinet restocking dates to be logged by the system, or the system may use hardware (e.g. sensors or scanners) inside the cabinet to detect when re-stocking has occurred. The re-stock log may be used to show that obligations to comply with legislated health and safety precautionary requirements are being met by the provision of a regularly re-stocked medical supply cabinet.

In some embodiments the system may interface with an independent time and/or date reference for logged events, to ensure the accuracy of time and date data. For example the system may receive co-ordinated universal time (UTC) data from an external source. Reference to an official source of time and date information may permit the log to be used for evidential purposes such as mandatory health and safety reporting and/or in court proceedings where health and safety liability is at issue.

The system preferably comprises or communicates with sensors 13 positioned on or inside the cabinet 1 to monitor the inventory levels therein, for example as shown in FIG. 2. Sensors 13 of the cabinet are used to be able to determine when an item is taken from the cabinet 1 or placed inside the cabinet 1. The sensors 13 are connected to a processor that may incorporate software to record at least one or more of:

a) what item(s) was/were taken,
b) the number of such items taken,
c) when the item(s) was/were taken, and
d) what item(s) was/were added to the cabinet,
e) the number of such items added,
f) when the item(s) was/were added, and
g) the cabinet being accessed but no items were taken or added.

It is preferable that the system will create a transaction record of anyone or more of these and may associate the image, footage and/or audio data, or fingerprint scanner/reader data with such a transaction record in order to provide information relating to the identity of the person associated with the particular record. In some embodiments the system may also be able to identify the person who is associated with that particular record, for example with reference to a data record of individuals known to use the cabinet (for example a database of known user fingerprints), or by access codes used to open the cabinet.

The sensors 13 allow a real time determination to be made of stock levels in the cabinet 1.

One or more of the sensors may be associated with the door 3 of the cabinet, and serve to detect when the door is opened and/or closed. One or more other sensors 13 may be associated with various compartments 5 and/or stock item bays 6 in the containment area 2.

In the preferred form, at least some of the sensors 13 are load sensors that determine the weight of stock bearing on the sensors 13. For example the sensors 13 may include micro switches, force sensing resistors or strain gauges. At least one item 14 or a collection of items that are preferably consolidated such as in a container or pack 15 are each located on a load sensor 13 in the containment area 2.

In the preferred form (for example as shown in FIGS. 2 and 3), there is a plurality of load sensors, each associated with a particular bay 6, or an item 14, or a collection of items. In the preferred form containers 15 are provided to carry a collection of items in a consolidated manner. A container 15 may contain a selection of items for use in treating burns or cuts etc. Alternatively, each item of the collection inside the container 15 may be of a standard size and/or type and/or weight, for example the container 15 may hold a plurality of bandages or a plurality of sticking plasters.

In the preferred embodiment each item 14 or container of items 15 is associated with one load sensor 13. A plurality of containers 15 of the same type may be associated with one load sensor 13 (for example as a stack of containers 15 as seen in FIG. 3). The sensor 13 can detect changes in the load it is receiving as items 14 or packs 15 are taken from or added to the cabinet. If a pack 15 is returned to the cabinet 1 with fewer items inside of it, the sensor will detect this too. Because the load on the sensor 13 prior to a transaction is known by the system, the detected loading subsequent to the transaction can be used to calculate how many items have been removed or added during the transaction.

The system may include or refer to a database containing pre-recorded information about the items stocked in the cabinet. For example, the database may contain a record indicating that the weight of a sticking plaster is twenty grams, and that there are ten plasters in a box. The system can use the output from a load sensor beneath the box of sticking plasters to detect that the box, having been removed from the cabinet, was forty grams lighter once replaced. The system can process this output to deduce that two plasters have been removed since the cabinet was last restocked with a new box of plasters, and that there are now only eight plasters left for others to use.

A person skilled in the art will appreciate that alternative means of counting or calculating the number of items removed or added could be employed, for example by the use of optical sensors or indexing mechanisms.

Other information may be input into the system and/or logged as part of the stock monitoring functionality. For example, there may be means for the system to monitor stock expiry dates. The system may, in turn, generate and output notifications when an item of stock has expired or is close to expiry.

For example, in some embodiments the expiry date for a particular item may be entered into the system as an external input (e.g. by a distributor/supplier at the time of re-stocking), or the system may be able to ascertain the expiry date by referring to a database containing product information associated with stocked items. As a further example, the system may be able to calculate the expected expiry date for a particular item based on a record of the date that the item was added to the cabinet. The record of the date that the item was added to the cabinet may have been entered as an input by the person who re-stocked the cabinet or may have been generated in response to sensor information from inside the cabinet indicating that new stock had been added.

In alternative embodiments there may be scanners or readers inside the cabinet 1 which are able to obtain expiry date information directly from one or more items. For example, a scanner may be able to scan visual indicia displayed on the package of an item which indicates its expiry date and relay this to the system for recording.

In preferred embodiments the cabinet 1 has a series of visual and/or audible indicators to provide information about the stock levels of supplies in the cabinet. An indicator 16, such as an LED light visible from the exterior of the cabinet 1, may be used to give an overall indication of inventory status. For example an LED light may glow green if all supplies are present in quantities above a specified minimum limit, orange if some of the supplies are nearing minimum levels, or red if there are supplies in the cabinet 1 which need replenishing.

Similarly there may be indicators 17 inside the cabinet 1 to indicate stock levels. Preferably each indicator 17 is associated with a particular stock item bay 6, so as to attract attention when that stock item needs replenishing and/or as a guide to the person carrying out the replenishment.

In some embodiments the sensors 13 can be used to detect when a particular item has been used and replaced inside the cabinet 1. In this embodiment, one or more indicators 18 may be used to indicate that an item is ‘used’ or ‘not sterile’. A person replenishing the cabinet 1 may need to reset the indicator 18 when clean or new equipment is added to the cabinet 1.

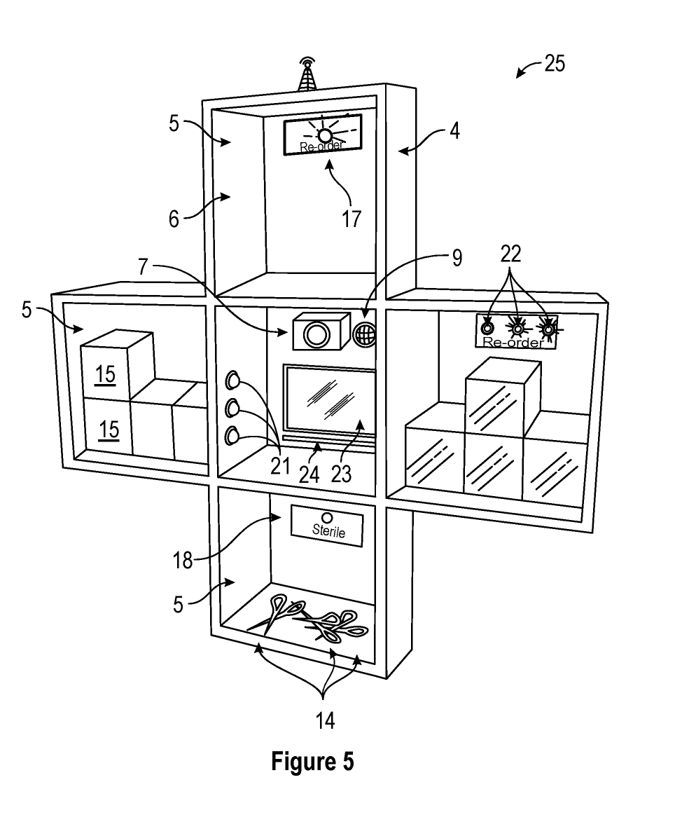

In some embodiments of the present invention, such as that shown in FIG. 5, there may be features of the cabinet 1 which assist a user in quickly and/or correctly identifying items contained by the cabinet that are appropriate for treating a particular type of injury. A user may be able to indicate a particular type of injury for which treatment is required (for example by way of a manual input device 21 such as a keyboard or a series of press buttons), and this may cause one or more of:

a) visual or audible indicators 22 inside the cabinet 1 to be activated in order to indicate the location of items which are suitable for treating that injury, and
b) a text, image or video display on a screen 23 which provides instructions or information pertaining to advisable treatment of the injury,
c) an audible message played through speaker 24 to convey information pertaining to advisable treatment of the injury.

For example, the user may be able to select from a series of press buttons 21, each press button being associated with a different type of injury selected from burns, cuts or contusions, in order to indicate that treatment for a burn injury is required. This input may cause LED indicators 22 positioned above particular bays 6 of the cabinet 1 to be lit, those particular bays 6 being ones which are designated to receive items suitable for treating burns. Also, or alternatively, the press button input may trigger a check list of points for the user to consider when treating the burn injury to be displayed on the screen 23. The screen 23 may be mounted inside the cabinet 1 and/or on a door of the cabinet.

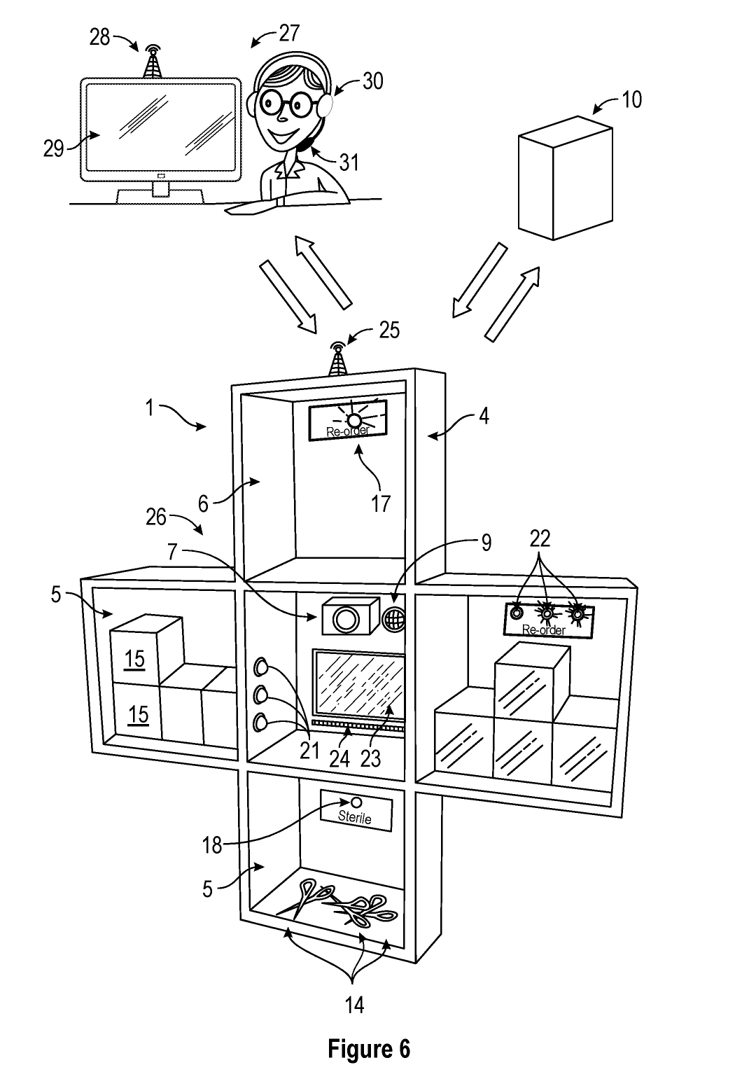

In further embodiments the cabinet 1 comprises a communication system 26 to facilitate communication between a user and a person who may be able to offer support and/or advice pertaining to injury treatment, for example as shown in FIG. 6. In preferred embodiments the communication system 26 includes a screen 23, speaker 24, microphone 9, optionally a camera 7, a transmitter for transmitting information from the microphone 9 and camera (if present) and a receiver for receiving information to be fed to the screen 23 and speaker 24. In some embodiments a single device 25 may act as both a transmitter and a receiver. Preferably the transmitter/receiver 25 is communicable with a further transmitter/receiver 28 at a remote station 27 so as to provide a video conferencing functionality whereby the cabinet user can communicate via a video link with a support person/advisor at the remote station 27.

The communication system 26 may include software for processing inputs and outputs from other components of the communication system 26 and from the remote station 27. Communication between the communication system 26 and the remote station 27 may be wireless or cabled, and preferably occurs via an internet or network connection.

For example, as shown in FIG. 6, the press button 21 may be pressed to initiate a video call that the treatment advisor stationed at the remote station 27 can answer. In some embodiments the cabinet mounted camera 7 (either as shown in FIG. 1 or in FIG. 5) may be used to obtain images or video feed of the injury that can be fed via the transmitter 25 to the remote station 27, and preferably the microphone 9 also picks up an audio feed from the injury site. Preferably the remote station 27 also includes a screen 29 to display the images or video feed, and a speaker 30 to project the audio feed. The remote station 27 preferably has a microphone 31 that the advisor can speak into, and/or a manual input (e.g. a keyboard) which the advisor can operate in order to provide advice to be relayed back to the cabinet user through the cabinet communication system 26. This may include the delivery of text, images or video to be displayed by the screen 23 and/or the delivery of audible information to be projected from the speaker 24.

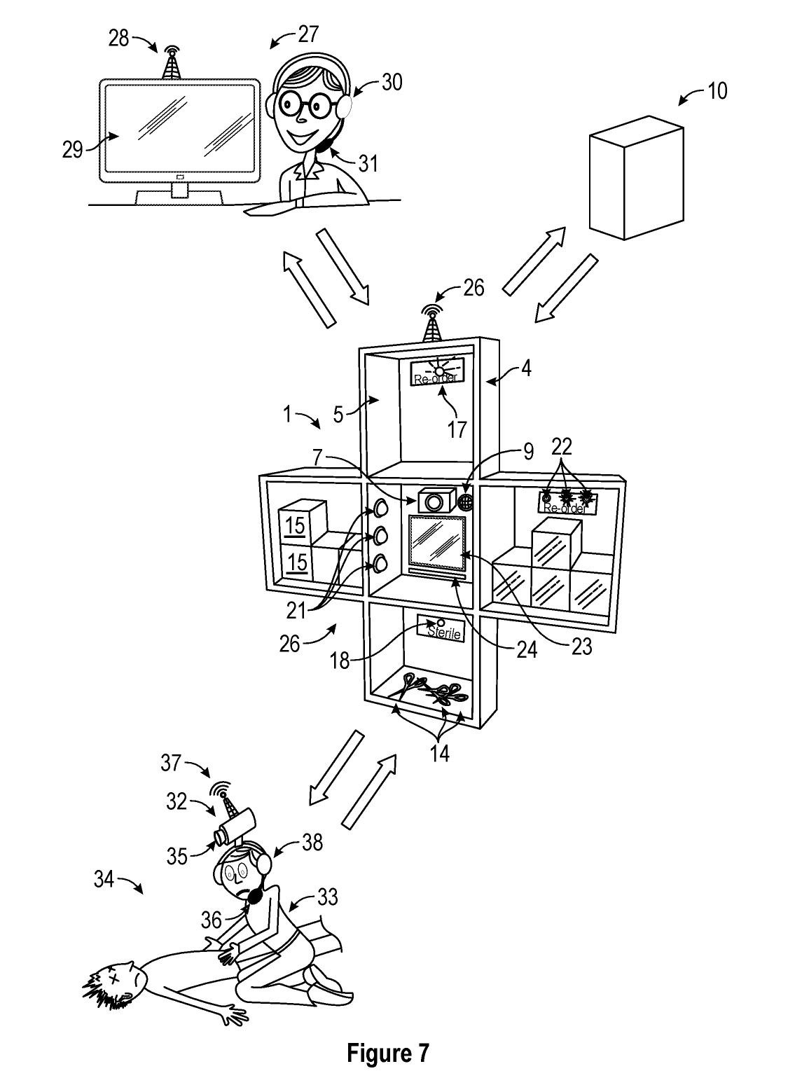

In some embodiments, for example as shown in FIG. 7, the cabinet 1 may further comprise a portable device 32 able to be worn or mounted upon a user 33 of the cabinet 1 during the treatment of an injury at an injury site 34 which is remote from the cabinet 1. Preferably the portable device 32 is adapted and configured to be worn or mounted on the head of the user 33, and for example, may take the form of a headset, goggles, glasses, a headband or a helmet.

The portable device 32 is preferably able to obtain visual and audible data, such as images, video and audio which can be used to assist a support person or advisor at a remote station 27 in assessing the injury that has occurred at the injury site 34 and providing advice on injury treatment accordingly. The advisor can then use the components of the remote station 27 (as described above in respect of FIG. 6) to communicate with the user 33 at the injury site 34, either by output which is sent directly to the device 32 worn by the user 33, or via the transmitter/receiver 25 of the cabinet 1.

Preferably the portable device 32 has at least one device for obtaining information at the injury site 34, and also comprises a transmitter 37 to transmit information from the injury site 34. The information obtaining device may be a camera 35 and/or a microphone 36. Preferably the portable device 32 further comprises one or more of a receiver 37 to receive inputs from the other components of the communication system 26 and/or the remote station 27, and an information delivery device to present or deliver information to the user carrying the device 32. A suitable information delivery device may be a speaker 38 to project an audio feed to the user 33, or a display screen may be used. Preferably the portable device 32 has an onboard power supply such as a battery.

Preferably the data obtaining device 32 has an onboard transmitter and/or receiver to enable communication with one or more other components of the communication system 26 of the cabinet. Preferably this communication is by way of a wireless connection. Preferably the transmitter 27 of the portable device 32 sends information to the receiver 25 of the communication system 26, or in some embodiments it may transmit directly to the receiver 28 of the remote station 27. Outputs transmitted from the remote station 27 and/or cabinet mounted components of the communication system 26 can be received by the receiver 37 of the device 32, and relayed to the user 33.

Preferably the portable device 32 has associated software which may be used to process data before it is transmitted or after it is received. The processing may occur before the information is relayed to the user by the information delivery device.

Preferably the data feeds to and from the portable device 32 are continuous and in real time.

For example, as shown in FIG. 7, a video stream captured by camera 35 at the injury site 34 can be sent to the remote station 27 and displayed on the screen 29 in real time. The advisor at the remote station 27 can use the video feed to assess the injury. The advisor can speak into the microphone 31 to provide instructions as to how the injury should be treated. The audio feed from the microphone 31 can be transmitted back to the user mounted device 32, and projected from the ear piece speaker 38 to the user 33.

Preferably the portable device 32 is stored inside cabinet 1, and when uplifted from the cabinet 1 for use, automatically establishes a connection with the communication system 26 of the cabinet. Preferably there are sensors of the cabinet 1 which can detect when the portable device 32 has been uplifted from the cabinet 1 for use. Preferably the connection is established when a sensor output indicates that the device 32 has been uplifted.

In some embodiments, such as those shown in FIGS. 6 and 7, one or more of the remote station 27, the communication system 26 and the portable device 32 comprise or communicate with a data storage medium, preferably an electronic data storage medium, and preferably being a storage medium of a data storage device 10 such as a network, hard drive or “the cloud”. Preferably information from the remote station 27 and/or communication system 26 and/or portable device 32 can be stored on the device 10 and accessed electronically to create a log of injury incidents, including information about the injury sustained and how it was treated. This can be used for reporting, auditing or evidential purposes, and may also be used in the production of training or educational resources.

Preferably the data storage device 10 is part of, or is integrated with, the previously described system for recording activity of the cabinet. Thus information regarding a particular injury incident can be associated with a transaction record, along with the information relating to the user of the cabinet for that transaction, the time and date of the transaction, and the items used from the cabinet in that transaction.

Where in the foregoing description reference has been made to elements or integers having known equivalents, then such equivalents are included as if they were individually set forth.

Although the invention has been described by way of example and with reference to particular embodiments, it is to be understood that modifications and/or improvements may be made without departing from the scope or spirit of the invention.

In addition, where features or aspects of the invention are described in terms of Markush groups, those skilled in the art will recognise that the invention is also thereby described in terms of any individual member or subgroup of members of the Markush group.

1. A monitored dispensary comprising:
a storage device to which multiple users have access, said storage device having a containment region inside of which items are stored, wherein the items contain or are items for use in medical treatment and
a monitoring system,
said monitoring system comprising at least
a) one or more detectors located inside the containment region and able to detect the removal and/or replacement and/or addition of items, and/or the weight of items prior to, during, and/or subsequent to any removal, replacement or addition transaction, and
b) one or more devices to obtain identity information derived from the user of the device at the time that the transaction was effected.

2. The monitored dispensary of claim 1, wherein said system further comprises a processor which is able to process the output of the detectors and the device in order to
i) create a record of each transaction, and
ii) associate the identity of the user with the transaction record, and
iii) monitor changes in the presence, absence and/or weight of items in the storage device from one transaction to the next.

3. The monitored dispensary of claim 2 which is also able report information generated by the functionalities of i), ii) and/or iii).

4. The monitored dispensary of claim 1, wherein said storage device is freely accessible by any user, said user needing no prior authorisation or qualification to be able to access the storage device.

5. The monitored dispensary of claim 1, wherein said containment region is divided into two or more compartments or bays, and wherein each of said compartments or bays is designated to receive either:
a) items suitable for treating a particular type or injury or for providing treatment in response to a particular type of accident, and/or
b) items falling into a particular category, said items having been categorised according to type, size, weight, use or some other physical characteristic.

6. The monitored dispensary of claim 5, wherein there is at least one detector associated with each compartment or bay of the containment area to detect the presence, absence or change in weight of the item or items held in each of the compartments.

7. The monitored dispensary of claim 1, wherein the storage device has or communicates with an input device which enables a user to specify a particular type of injury, wherein said input results in indicators inside the containment region indicating the location of items which may be useful in treating the specified type of injury.

8. The monitored dispensary of claim 1, wherein monitoring changes in the presence, absence and/or weight of items in the storage device from one transaction to the next results in:
a) the reporting of stock levels of items contained inside the device, and/or
b) the generating of notifications as items are taken, and/or as the stock levels of particular types or categories of item fall below a predetermined threshold level.

9. The monitored dispensary of claim 1, wherein there is real time reporting of stock levels of items contained inside the device.

10. The monitored dispensary of claim 1, wherein said system further comprises a processor which is able to process the output of the detectors and/or the device for obtaining identity information in order to determine one or more of:
a) what item(s) was/were taken,
b) the number of such items taken,
c) when the item(s) was/were taken, and
d) what item(s) were added to the cabinet,
e) the number of such items added,
f) when the item(s) were added, and
g) the cabinet being accessed but no items were taken or added.

11. The monitored dispensary of claim 1, wherein there are indicators located on or inside the storage device to provide information about the stock level and/or sterility of one or more of the items stocked in the cabinet.

12. The monitored dispensary of claim 1, wherein said storage device has a communication system configured and adapted for communication with a remote station.

13. The monitored dispensary of claim 12, wherein the communication system further comprises a portable device which can be used away from the cabinet, said portable device comprising at least an information obtaining device and an information delivery device.

14. The monitored dispensary of claim 12, wherein said monitoring system is able to associate information processed by the communication system with a particular transaction record.

15. A storage device to which multiple users have access, said storage device having a containment region inside of which items are stored, wherein the items contain or are items for use in medical treatment, and
one or more detectors located inside the containment region and able to detect the removal and/or replacement and/or addition of items, and/or the weight of items subsequent to any replacement or addition transaction,
and wherein the storage device further comprises one or more devices to obtain identity information derived from the user of the cabinet at the time that the transaction was effected.

16. The storage device of claim 15, wherein said storage device is a cabinet.

17. The storage device of claim 15, wherein said storage device is freely accessible by any user, said user needing no prior authorisation or qualification to be able to access the storage device.

18. The storage device of claim 15, wherein said containment region is divided into two or more compartments or bays, and wherein each of said compartments or bays is designated to receive either:
a) items suitable for treating a particular type or injury or for providing treatment in response to a particular type of accident, and/or
b) items falling into a particular category, said items having been categorised according to type, size, weight, use or some other physical characteristic.

19. The storage device of claim 18, wherein there is at least one detector associated with each compartment or bay of the containment area to detect the presence, absence or change in weight of the item or items held in each of the compartments or bays.

20. The storage device of claim 19, wherein at least some of said detectors are load sensors.

21. The storage device of claim 15, wherein said storage device has or communicates with an input device which enables a user to specify a particular type of injury, wherein said input results in indicators inside the containment region indicating the location of items which may be useful in treating the specified type of injury.

22. The storage device of claim 15, wherein said device to obtain identity information derived from the user is a camera or optical device.

23. The storage device of claim 15, wherein there are indicators located on or inside the storage device to provide information about the stock level and/or sterility of one or more of the items stocked in the cabinet.
